(19) United States
(12) Patent Application Publication
(10) Pub. No.: US 2023/0404951 A1
(43) Pub. Date: Dec. 21, 2023

(54) Pet Food Compositions

(71) Applicant: Hill's Pet Nutrition, Inc., Topeka, KS (US)

(72) Inventor: Eden EPHRAIM, Lawrence, KS (US)

(73) Assignee: Hill's Pet Nutrition, Inc., Topeka, KS (US)

(21) Appl. No.: 18/335,201

(22) Filed: Jun. 15, 2023

Related U.S. Application Data
(60) Provisional application No. 63/352,461, filed on Jun. 15, 2022.

(51) Int. Cl. A61K 31/19; A23K 50/40; A23K 20/158; A23K 20/147; A23K 20/163; A61K 9/00

(52) U.S. Cl. CPC A61K 31/19; A23K 50/40; A23K 20/158; A23K 20/147; A23K 20/163; A61K 9/0056

(57) ABSTRACT

Pet food compositions and methods for improving the health of pets are disclosed herein. In accordance with one aspect, provided is a pet food composition comprising a fat; from about 15 to about 55 wt. % of a protein; from about 0.1 to about 5 wt. % of a crude fiber; and optionally, a carbohydrate, wherein the pet food composition has a weight ratio of the protein to the crude fiber of from about 25:1 to about 50:1, and wherein all weight percentages are based on the total weight of the pet food composition. In some embodiments of the pet food composition, the weight ratio of the protein to the crude fiber is from about 28:1 to about 45:1, preferably about 30:1 to about 40:1, preferably about 30:1 to about 38:1, or preferably about 32:1 to about 36:1.

### CROSS-REFERENCE TO RELATED PATENT APPLICATIONS

This application claims priority to U.S. Appl. No. 63/352,461, titled PET FOOD COMPOSITIONS and filed Jun. 15, 2022, the content of which is incorporated herein by reference in its entirety for all purposes.

### BACKGROUND

Animals, particularly companion pet animals (e.g., dogs and cats), suffer from a wide variety of health issues as they age. Previous attempts to address these issues focused on treating being over-weight, or in extreme cases being obese. Specifically, food compositions were specifically tailored to reduce caloric intake or affect metabolism solely to reduce the weight of the animal. Separate from controlling the diet of an animal, these animals also suffer from separate age-related gastrointestinal health issues.

As an additional difficulty for producing pet food compositions that may be tailored to modify caloric intake and/or address gastrointestinal health issues, determining the appropriate level of dietary protein for the respective animal is challenging. Increasing the dietary protein levels in an animals food may help prevent sarcopenia through preservation of lean mass, and to induce food satiety that will bolster compliance with weight management diets, but may lead to an increased amount of dietary protein bypassing digestion to be putrefied by resident commensal gut bacteria. Microbial protein putrefaction products can be detrimental to the progression of renal disease in renal compromised pets.

There is an ongoing desire for improved pet food compositions that can provide health benefits to domestic pets.

### BRIEF SUMMARY

This summary is intended merely to introduce a simplified summary of some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

Aspects of the invention are directed to pet food compositions and methods for improving the health of pets. In accordance with a first aspect of the invention, provided is a pet food composition comprising a fat; from about 15 to about 55 wt. % of a protein; from about 0.1 to about 5 wt. % of a crude fiber; and optionally, a carbohydrate, wherein the pet food composition has a weight ratio of the protein to the crude fiber of from about 25:1 to about 50:1, and wherein all weight percentages are based on the total weight of the pet food composition. In some embodiments of the pet food composition, the weight ratio of the protein to the crude fiber is from about 28:1 to about 45:1, preferably about 30:1 to about 40:1, preferably about 30:1 to about 38:1, or preferably about 32:1 to about 36:1.

According to another aspect of the invention, provided is a pet food composition comprising from about 8 to about 50 wt. % of a fat; from about 15 to about 55 wt. % of a protein; from about 0.1 to about 5 wt. % of crude fiber; and an amount up to about 65 wt. % of a carbohydrate, wherein the pet food composition has a weight ratio of the protein to the crude fiber of from about 25:1 to about 50:1, and wherein all weight percentages are based on the total weight of the pet food composition.

In yet a further embodiment, a method is provided for reducing metabolites associated with protein putrefaction in a feline. The method typically comprises reducing one or more proteolytic metabolites and increasing one or more short chain fatty acid in a feline by administering a pet food composition described herein to the feline. The one or more short chain fatty acid increased by the method is preferably butyrate (butyrylglycine). In some embodiments, the one or more proteolytic metabolites comprises pyrraline, N6-carboxymethyllysine, N6-carboxyethyllysine, methionine sulfoxide, pseudouridine, or a combination of two or more thereof.

The method may further comprise feeding the feline the pet food composition at least once a day, preferably at least twice a day, or preferably at least three times a day. Additionally or alternatively, the feline may be fed the pet food composition for 1 or more days, preferably 5 or more days, preferably 7 or more days, preferably 10 or more days, preferably 14 or more days, preferably 30 or more days, or preferably 42 or more days.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges such as 2-5, 3-5, 2-3, 2-4, 1-4, etc. The term “about” when referring to a number means any number within a range of 10% of the number. For example, the phrase “about 2.0 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means percent by weight with respect to the pet food composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the pet food compositions of the instant disclosure can be free or essentially free of all components and elements positively recited throughout the instant disclosure. In some instances, the pet food compositions of the present disclosure may be substantially free of non-incidental amounts of the ingredient(s) or compound(s) described herein. A non-incidental amount of an ingredient or compound is the amount of that ingredient or compound that is added into the pet food composition by itself. For example, a pet food composition may be substantially free of a non-incidental amount of an ingredient or compound, although such ingredient(s) or compound(s) may be present as part of a raw material that is included as a blend of two or more compounds. Substantially free, unless other defined or described herein, typically refers to an ingredient or compound in an amount of about 2 wt. % or less, about 1.5 wt. % or less, about 1 wt. % or less, about 0.5 wt. % or less, about 0.1 wt. % or less, or about 0.05 wt. % or less, or about 0.01 wt. % or less, based on the total weight of the pet food composition on a dry matter basis.

Some of the various categories of components identified may overlap. In such cases where overlap may exist and the pet food composition includes both components (or the composition includes more than two components that overlap), an overlapping compound does not represent more than one component. For example, certain components or ingredients may be characterized as both a grain and a rice. If a particular pet food care composition recites both a grain and a rice, an ingredient and/or compound that may be characterized as both a grain and a rice will serve only as either a grain or a rice—not both.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (i.e., farm cats), pet cats, ferrets, birds, reptiles, rabbits, and fish.

To the extent that food and food ingredient contain water/moisture, the dry matter represents everything in the sample other than water including, for example, protein, fiber, fat, carbohydrates, minerals, etc. Dry matter weight is the total weight minus the weight of any water. The skilled artisan would readily recognize and understand nutritional amounts and percentages expressed as dry matter amounts, dry matter weights and dry matter percentages.

Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day.

“Daily nutritional intake” and “total nutritional intake per day” refer to dry matter intake per day. That is, water weight is not included in calculating the amount of nutrition consumed per day. To calculate percent of an ingredient of total daily intake on a dry matter basis, water is removed from the total intake to give total daily dry matter intake and the percentage of the ingredient is based on amount of ingredient present as dry matter.

As used herein, an “ingredient” refers to any component of a pet food composition. The term “nutrient” refers to a substance that provides nourishment and thus has a nutrient value. In some cases, an ingredient may comprise more than one “nutrient,” for example, a composition may comprise corn comprising important nutrients including both protein and carbohydrate.

Aspects of the invention are directed to pet food compositions and methods for improving the health of pets. The pet food compositions may be specifically formulated for felines and/or canines. In some aspects, the pet food compositions contain higher amounts of protein, while avoiding heightened levels of gut microbial protein putrefaction typical with conventional pet food compositions containing such levels of dietary protein. For instance, certain embodiments of the pet food compositions that contain certain proteins and fibers in particular ratios achieve unexpected reductions in reduced fecal putrefaction and oxidative metabolites, while simultaneously increasing butyrate from consumption of such pet food compositions.

In one aspect of the invention, provided is a pet food composition comprising a fat; from about 15 to about 55 wt. % of a protein; from about 0.1 to about 5 wt. % of a crude fiber; and optionally, a carbohydrate, wherein the pet food composition has a weight ratio of the protein to the crude fiber of from about 25:1 to about 50:1, and all weight percentages are based on the total weight of the pet food composition.

In some embodiments, the pet food composition is formulated to have a weight ratio of the protein to the crude fiber of from about 25:1 to about 47:1, about 25:1 to about 45:1, about 25:1 to about 43:1, about 25:1 to about 41:1, about 25:1 to about 39:1, about 25:1 to about 37:1, about to about 35:1; from about 27:1 to about 50:1, about 27:1 to about 47:1, about 27:1 to about about 27:1 to about 43:1, about 27:1 to about 41:1, about 27:1 to about 39:1, about 27:1 to about 37:1, about 27:1 to about 35:1; from about 29:1 to about 50:1, about 29:1 to about 47:1, about 29:1 to about 45:1, about 29:1 to about 43:1, about 29:1 to about 41:1, about 29:1 to about 39:1, about 29:1 to about 37:1, about 29:1 to about 35:1; from about 31:1 to about 50:1, about 31:1 to about 47:1, about 31:1 to about 45:1, about 31:1 to about 43:1, about 31:1 to about 41:1, about 31:1 to about 39:1, about 31:1 to about 37:1, about 31:1 to about 35:1; from about 33:1 to about about 33:1 to about 47:1, about 33:1 to about 45:1, about 33:1 to about 43:1, about 33:1 to about 41:1, about 33:1 to about 39:1, about 33:1 to about 37:1, or about 33:1 to about 35:1, including any ranges or subranges thereof.

Suitable components, such as those listed below, may be included or excluded from the formulations for the pet food compositions depending on the specific combination of other ingredients and the form of the pet food compositions. In some embodiments, the pet food compositions disclosed herein may be in the form of a stand-alone pet food, as a supplement to pet food, as a pet food treat, or the like.

The pet food compositions are formulated to include fat in an amount that may vary, but typically is in the range of about 8 to about 50 wt. %, endpoints included, based on the total weight of the pet food composition on a dry matter basis. For example, the pet food composition may include fat in an amount ranging from about 10 to about 50 wt. %, about 12 to about 50 wt. %, about 14 to about 50 wt. %, about 16 to about 50 wt. %, about 18 to about 50 wt. %, about 20 to about 50 wt. %, about 22 to about 50 wt. %, about 24 to about 50 wt. %; from about 8 to about 40 wt. %, about to about 40 wt. %, about 12 to about 40 wt. %, about 14 to about 40 wt. %, about 16 to about 40 wt. %, about 18 to about 40 wt. %, about 20 to about 40 wt. %, about 22 to about 40 wt. %, about 24 to about 40 wt. %; from about 8 to about 35 wt. %, about 10 to about 35 wt. %, about 12 to about 35 wt. %, about 14 to about 35 wt. %, about 16 to about 35 wt. %, about 18 to about 35 wt. %, about 20 to about 35 wt. %, about 22 to about 35 wt. %, about 24 to about 35 wt. %; about 8 to about 30 wt. %, about 10 to about 30 wt. %, about 12 to about 30 wt. %, about 14 to about 30 wt. %, about 16 to about 30 wt. %, about 18 to about 30 wt. %, about 20 to about 30 wt. %, about 22 to about 30 wt. %, about 24 to about 30 wt. %; from about 8 to about 27 wt. %, about 10 to about 27 wt. %, about 12 to about 27 wt. %, about 14 to about 27 wt. %, about 16 to about 27 wt. %, about 18 to about 27 wt. %, about 20 to about 27 wt. %, about 22 to about 27 wt. %; from about 8 to about 24 wt. %, about to about 24 wt. %, about 12 to about 24 wt. %, about 14 to about 24 wt. %, about 16 to about 24 wt. %, about 18 to about 24 wt. %, about 20 to about 24 wt. %; from about 8 to about 22 wt. %, about to about 22 wt. %, about 12 to about 22 wt. %, about 14 to about 22 wt. %, about 16 to about 22 wt. %, about 18 to about 22 wt. %; from about 8 to about 22 wt. %, about 10 to about 22 wt. %, about 12 to about 22 wt. %, about 14 to about 22 wt. %, about 16 to about 22 wt. %, about 18 to about 22 wt. %; from about 8 to about 20 wt. %, about 10 to about 20 wt. %, about 12 to about 20 wt. %, about 14 to about 20 wt. %, about 16 to about 20 wt. %, including ranges and subranges thereof, based on the total weight of the pet food composition on a dry matter basis.

The term “fat” generally refers to a lipid or mixture of lipids that may generally be a solid or a liquid at ordinary room temperatures (e.g., 25° C.) and pressures (e.g., 1 atm). In some instances, the fat may be a viscous liquid or an amorphous solid at standard room temperature and pressure. The fat may be incorporated completely within the food composition, deposited on the outside of the pet food composition, or a mixture of the two methods. In some embodiments, the pet food compositions further include an effective amount of one or more substances selected from the group consisting of glucosamine, chondroitin, chondroitin sulfate, methylsulfonylmethane (“MSM”), creatine, antioxidants, Perna canaliculata, and mixtures thereof.

The fat may comprise dietary fats, such as triglycerides. In some embodiments, the triglyceride may comprise about 20 to about 100%, about 40 to about 100%, about 50 to about 100%, about 60 to about 100%, about 70 to about 100%, about 80 to about 100%, about 90 to about 100%, of the total amount of fat in the pet food composition. In further embodiments, the triglyceride comprises about 20 to about 95%, about 40 to about 95%, about 50 to about 95%, about 60 to about 95%, about 70 to about 95%, about 80 to about 95%, about 90 to about 95%, of the total amount of fat in the pet food composition. In additional embodiments, the triglyceride comprises about 20 to about 90%, about 40 to about 90%, about 50 to about 90%, about 60 to about 90%, about 70 to about 90%, about 80 to about 90%, of the total amount of fat in the pet food composition. In yet further embodiment, the triglyceride comprises about 20 to about 80%, about 40 to about 80%, about 50 to about 80%, about 60 to about 80%, about 70 to about 80%, of the total amount of fat in the pet food composition.

The triglyceride may include one or more constituents that comprise a fatty acid(s) component/moiety. For example, the triglyceride may include one, two, or three aplanatic chains that are selected from fatty acid component/moieties. Non-limiting examples of fatty acid components include, but are not limited to, omega-3 fatty acids, omega-6 fatty acids, lauric acid, myristic acid, palmitic acid, palmitoleic acid, margaric acid, margaroleic acid, stearic acid, oleic acid, stearidonic acid, gadoleic acid, behenic acid, erucic acid, docosatetra acid, and a combination of two or more thereof. The fatty acid(s) components may be a polyunsaturated fatty acid, such as an omega-3 fatty acid, an omega-6 fatty acid, or a combination of two or more thereof. Non-limiting examples of omega-3 fatty acid components include those selected from linolenic acid, stearidonic acid, eicosatetraenoic acid, eicosapentaenoic acid, docosapentaenoic acid, docosahexaenoic acid, and a combination of two or more thereof.

Additionally or alternatively, the triglyceride may include a polyunsaturated fatty acid component selected from omega-6 fatty acids. Examples of omega-6 fatty acid component include linolenic acid, calendic acid, eicosadienoic acid, arachidonic acid, docosadienoic acid, adrenic acid, osbond acid, tetracosatetraenoic acid, tetracosapentaenoic acid, or a combination of two or more thereof. In some embodiments, the polyunsaturated fatty acid component is selected from linolenic acid, arachidonic acid, and a combination of two or more thereof.

The triglyceride may be selected from triglycerides having at least one aliphatic carbon chain comprised of 6 to 10 carbons. For example, the triglyceride may have one, two, or three aliphatic carbons chains of 6 to 10 carbons. In some embodiments, the triglyceride has an aliphatic carbon chain of 6 carbons, 7 carbons, 8 carbons, 9 carbons, and/or 10 carbons. For example, the aliphatic carbon chain of the medium chain triglyceride may include from 6 to 9 carbons, 6 to 8 carbons, 6 or 7 carbons; 7 to 10 carbons, 7 to 9 carbons, 7 or 8 carbons; 8 to 10 carbons, or 8 or 9 carbons, or 9 or 10 carbons. In some embodiments, the triglyceride(s) comprises an aliphatic carbon chain having 6 carbons, 8 carbons, or 10 carbons. For example, the triglyceride may have an aliphatic carbon chain selected from caprylate, caprate, and/or decanoate.

Fat can be supplied by any of a variety of sources known by those skilled in the art, including meat, meat by-products, canola oil, fish oil such as anchovy oil and menhaden oil, and plants. Meat fat sources include poultry fat, turkey fat, pork fat, lard, tallow, and beef fat. Plant fat sources include wheat, flaxseed, rye, barley, rice, sorghum, corn, oats, millet, wheat germ, corn germ, soybeans, peanuts, and cottonseed, as well as oils derived from these and other plant fat sources such as corn oil, soybean oil, cottonseed oil, palm oil, palm kernel oil, linseed oil, canola oil, rapeseed oil, and/or olestra.

In some cases, the fat in the compositions is crude fat. Crude fat may be included into the compositions in the amounts disclosed above with respect of the total fat, such as from about 8 to about 50 wt. %, based on the total weight of the pet food composition on a dry matter basis. In some embodiments, the pet food composition comprises crude fat in an amount of about 10 to about 40 wt. %, about 12 to about 35 wt. %, about 14 to about 30 wt. %, about 16 to about 24 wt. %, based on the total weight of the pet food composition on a dry matter basis. In some cases, it may be preferable that about 50 wt. % or more, about 60 wt. % or more, about 70 wt. % or more, about wt. % or more, or about 90 wt. % or more of the total fat is obtained from an animal source. Alternatively, about 50 wt. % or more, about 60 wt. % or more, about 70 wt. % or more, about 80 wt. % or more, or about 90 wt. % or more of the total fat may be obtained from a plant source.

The pet food compositions typically include protein in an amount ranging from about 15 to about 55 wt. %, based on the total weight of the pet food composition on a dry matter basis. In some instances, the total amount of protein in the pet food composition is in a range from about to about 50 wt. %, about 15 to about 48 wt. %, about 15 to about 46 wt. %, about 15 to about 44 wt. %, about 15 to about 42 wt. %, about 15 to about 40 wt. %, about 15 to about 38 wt. %, about 15 to about 36 wt. %, about 15 to about 34 wt. %; from about 20 to about 55 wt. %, about 20 to about wt. %, about 20 to about 48 wt. %, about 20 to about 46 wt. %, about 20 to about 44 wt. %, about to about 42 wt. %, about 20 to about 40 wt. %, about 20 to about 38 wt. %, about 20 to about 36 wt. %, about 20 to about 34 wt. %; from about 25 to about 55 wt. %, about 25 to about 50 wt. %, about 25 to about 48 wt. %, about 25 to about 46 wt. %, about 25 to about 44 wt. %, about 25 to about 42 wt. %, about 25 to about 40 wt. %, about 25 to about 38 wt. %, about 25 to about 36 wt. %, about 25 to about 34 wt. %; from about 30 to about 55 wt. %, about 30 to about 50 wt. %, about 30 to about 48 wt. %, about 30 to about 46 wt. %, about 30 to about 44 wt. %, about 30 to about 42 wt. %, about 30 to about 40 wt. %, about 30 to about 38 wt. %, or about 30 to about 36 wt. %, including ranges and subranges therebetween, based on the total weight of the pet food composition on a dry matter basis.

The protein of the pet food composition comprises one or more amino acids selected from Tryptophan, Taurine, Histidine, Carnitine, Carnosine, Alanine, Cysteine, Arginine, Methionine (including DL-methionine, and L-methionine), Tryptophan, Lysine, Asparagine, Aspartate (Aspartic acid), Phenylalanine, Valine, Threonine, Isoleucine, Histidine, Leucine, Glycine, Glutamine, Taurine, Tyrosine, Homocysteine, Ornithine, Citruline, Glutamate (Glutamic acid), Proline, and/or Serine, and a combination of two or more thereof. The pet food composition may comprise two or more amino acids. For instance, the pet food composition may include two or more, three or more, four or more, five or more, six or more, seven or more, eight or more amino acids. In some embodiments, the pet food composition includes glycine and proline, and optionally one or more additional amino acids.

In some cases, the one or more amino acid(s) may comprise essential amino acids. Essential amino acids are amino acids that cannot be synthesized de novo, or in sufficient quantities by an organism and thus must be supplied in the diet. Essential amino acids vary from species to species, depending upon the organism's metabolism. For example, it is generally understood that the essential amino acids for dogs and cats (and humans) are phenylalanine, leucine, methionine, lysine, isoleucine, valine, threonine, tryptophan, histidine and arginine. In addition, taurine, while technically not an amino acid but a derivative of cysteine, is an essential nutrient for cats.

A portion of the protein in the composition may be digested (pre-hydrolyzed) protein. For example, the composition may include an amount of protein, where about 40 wt. % or more, about wt. % or more, about 60 wt. % or more, about 70 wt. % or more, about 80 wt. % or more, about wt. % or more, about 95 wt. % or more, about 98 wt. % or more, or about 99 wt. % or more of the protein is digested (pre-hydrolyzed) protein. In some embodiments, e.g., when the composition desirable promotes weight loss, the portion of protein that is digested (e.g., hydrolyzed) protein is about 60 wt. % or less, about 50 wt. % or less, about 40 wt. % or less, about 30 wt. % or less, about wt. % or less, or about 10 wt. % or less, based on the total amount of protein in the pet food composition on a dry matter basis. In further embodiment, the amount of protein that is digested (e.g., hydrolyzed) protein is about 10 to about 99 wt. %, about 10 to about 95 wt. %, about 10 to about 90 wt. %, about 10 to about 70 wt. %, about 10 to about 50 wt. %, about 10 to about 30 wt. %; about 30 to about 99 wt. %, about 30 to about 95 wt. %, about 30 to about 90 wt. %, about 30 to about 70 wt. %, about 30 to about 50 wt. %; about 50 to about 99 wt. %, about 50 to about 95 wt. %, about 50 to about 90 wt. %, about 50 to about 70 wt. %; or about 70 to about 99 wt. %, about 70 to about 95 wt. %, about 70 to about 90 wt. %, including ranges and subranges therein, based on the total amount of protein in the pet food composition on a dry matter basis.

Protein may be supplied by any of a variety of sources known by those of ordinary skill in the art including plant sources, animal sources, microbial sources or a combination of these. For example, animal sources may include meat, meat-by products, seafood, dairy, eggs, etc. Meats, for example, may include animal flesh such as poultry, fish, and mammals including cattle, pigs, sheep, goats, and the like. Meat by-products may include, for example, lungs, kidneys, brain, livers, stomachs and intestines. Plant protein includes, for example, soybean, cottonseed, and peanuts. Microbial sources may be used to synthesize amino acids (e.g., lysine, threonine, tryptophan, methionine) or intact protein such as protein from sources listed below.

Examples of protein or protein ingredients may comprise chicken meals, chicken, chicken by-product meals, lamb, lamb meals, turkey, turkey meals, beef, beef by-products, viscera, fish meal, enterals, kangaroo, white fish, venison, soybean meal, soy protein isolate, soy protein concentrate, corn gluten meal, corn protein concentrate, distillers dried grains, and/or distillers dried grain solubles and single-cell proteins, for example yeast, algae, and/or bacteria cultures.

The protein can be intact, completely hydrolyzed, or partially hydrolyzed. The protein content of foods may be determined by any number of methods known by those of skill in the art, for example, as published by the Association of Official Analytical Chemists in Official Methods of Analysis (“OMA”), method 988.05. The amount of protein in a composition disclosed herein may be determined based on the amount of nitrogen in the composition according to methods familiar to one of skill in the art.

The pet food compositions typically include from about 0.1 to about 5 wt. % of a crude fiber, based on the total weight of the pet food composition on a dry matter basis. In some embodiments, the amount of crude fiber present in the pet food compositions is from about 0.1 to about 4.5 wt. %, about 0.1 to about 4 wt. %, about 0.1 to about 3.5 wt. %, about 0.1 to about 3 wt. %, about 0.1 to about 2.5 wt. %, about 0.1 to about 2 wt. %, about 0.1 to about 1.5 wt. %, about 0.1 to about 1 wt. %; from about 0.5 to about 5 wt. %, about 0.5 to about 4.5 wt. %, about 0.5 to about 4 wt. %, about 0.5 to about 3.5 wt. %, about 0.5 to about 3 wt. %, about 0.5 to about 2.5 wt. %, about to about 2 wt. %, about 0.5 to about 1.5 wt. %, about 0.5 to about 1 wt. %; from about 1 to about wt. %, about 1 to about 4.5 wt. %, about 1 to about 4 wt. %, about 1 to about 3.5 wt. %, about 1 to about 3 wt. %, about 1 to about 2.5 wt. %, about 1 to about 2 wt. %, about 1 to about 1.5 wt. %; from about 2 to about 5 wt. %, about 2 to about 4.5 wt. %, about 2 to about 4 wt. %, about 2 to about 3.5 wt. %, about 2 to about 3 wt. %, about 2 to about 2.5 wt. %; from about 3 to about 5 wt. %, about 3 to about 4.5 wt. %, about 3 to about 4 wt. %, about 3 to about 3.5 wt. %; from about 4 to about 5 wt. %, or about 4 to about 4.5 wt. %, including ranges and subranges thereof, based on the total weight of the pet food composition on a dry matter basis.

As mentioned above, the pet food composition may be formulated to have a weight ratio of the protein to the crude fiber that is from about 25:1 to about 47:1, about 25:1 to about 45:1, about 25:1 to about 43:1, about 25:1 to about 41:1, about 25:1 to about 39:1, about 25:1 to about 37:1, about 25:1 to about 35:1; from about 27:1 to about 50:1, about 27:1 to about 47:1, about 27:1 to about 45:1, about 27:1 to about 43:1, about 27:1 to about 41:1, about 27:1 to about 39:1, about 27:1 to about 37:1, about 27:1 to about 35:1; from about 29:1 to about 50:1, about 29:1 to about 47:1, about 29:1 to about 45:1, about 29:1 to about 43:1, about 29:1 to about 41:1, about 29:1 to about 39:1, about 29:1 to about 37:1, about 29:1 to about 35:1; from about 31:1 to about 50:1, about 31:1 to about 47:1, about 31:1 to about 45:1, about 31:1 to about 43:1, about 31:1 to about 41:1, about 31:1 to about 39:1, about 31:1 to about 37:1, about 31:1 to about 35:1; from about 33:1 to about 50:1, about 33:1 to about 47:1, about 33:1 to about 45:1, about 33:1 to about 43:1, about 33:1 to about 41:1, about 33:1 to about 39:1, about 33:1 to about 37:1, or about 33:1 to about 35:1, including any ranges or subranges thereof.

Additionally, the pet food composition may be formulated to have a total amount of fiber of from about 0.1 to about 15 wt. %, based on the total weight of the pet food composition on a dry matter basis. For example, the total amount of fiber in the pet food composition may be from about 0.1 to about 12 wt. %, about 0.1 to about 9 wt. %, about 0.1 to about 7 wt. %, about 0.1 to about 5 wt. %, about 0.1 to about 4 wt. %, about 0.1 to about 3 wt. %, about 0.1 to about 2 wt. %; from about 1 to about 12 wt. %, about 1 to about 9 wt. %, about 1 to about 7 wt. %, about 1 to about 5 wt. %, about 1 to about 4 wt. %, about 1 to about 3 wt. %, about 1 to about 2 wt. %; from about 2 to about 12 wt. %, about 2 to about 9 wt. %, about 2 to about 7 wt. %, about 2 to about 5 wt. %, about 2 to about 4 wt. %, about 2 to about 3 wt. %; from about 4 to about 12 wt. %, about 4 to about 9 wt. %, about 4 to about 7 wt. %, about 4 to about 5 wt. %; from about 6 to about 12 wt. %, about 6 to about 9 wt. %, about 6 to about 7 wt. %; from about 8 to about 12 wt. %, about 8 to about 9 wt. %; or from about 10 to about 12 wt. %, including any ranges or subranges thereof, based on the total weight of the pet food composition on a dry matter basis.

In some embodiments, crude fiber comprises the majority of the total amount of fiber. In at least one embodiment, the crude fiber comprises about 55 wt. % or more, about 65 wt. % or more, about 75 wt. % or more, about 80 wt. % or more, about 85 wt. % or more, about 90 wt. % or more, about 95 wt. % or more, or about 98 wt. % or more of the total amount of fiber in the pet food composition, based on the total weight of the pet food composition on a dry matter basis. In a further embodiment, the total amount of fiber consists essentially of or consists of crude fiber.

The total amount of fiber present in the pet food composition generally comprises an amount of crude fiber and dietary fiber. The amount of crude fiber and/or dietary fiber may be present in the pet food compositions in any of the above amounts disclosed for the total amount of fiber. Crude fiber includes indigestible components contained in cell walls and cell contents of plants such as grains, e.g., hulls of grains such as rice, corn, and beans.

Dietary fiber refers to components of a plant that are resistant to digestion by an animal's digestive enzymes. Dietary fiber includes soluble fiber and insoluble fiber. Soluble fibers are resistant to digestion and absorption in the small intestine and undergo complete or partial fermentation in the large intestine, e.g., beet pulp, guar gum, chicory root, psyllium, pectin, blueberry, cranberry, squash, apples, oats, beans, citrus, barley, or peas. Insoluble fibers can be supplied by any of a variety of sources, including, for example, cellulose, whole-wheat products, wheat oat, corn bran, flax seed, grapes, celery, green beans, cauliflower, potato skins, fruit skins, vegetable skins, peanut hulls, and soy fiber. Crude fiber includes indigestible components contained in cell walls and cell contents of plants such as grains, for example, hulls of grains such as rice, corn, and beans.

In some embodiments, the pet food composition has a weight ratio of insoluble fiber to soluble fiber from about 20:1 to about 8:1. For example, the pet food composition may have a weight ratio of insoluble fiber to soluble fiber from about 18:1 to about 8:1, about 16:1 to about 8:1, about 14:1 to about 8:1, about 12:1 to about 8:1, about 10:1 to about 8:1. The pet food composition may have a weight ratio of insoluble fiber to soluble fiber of about 11:1.

The pet food composition may further comprise ash. As described herein, ash consists of compounds that are not organic or water, generally produced by combustion of biological materials. The ash may be present in the pet food composition in an amount ranging from about 1 to about 10 wt. %, based on the total weight of the food composition on a dry weight basis, including all amounts and sub-ranges there-between. In some embodiment, the ash may be present in the food composition in an amount ranging from about 1 to about 8 wt. %, about 1 to about 6 wt. %, about 1 to about 5 wt. %, about 1 to about 4 wt. %, about 1 to about 3 wt. %, about 1 to about 2 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %; from about 5 to about 10 wt. %, about 5 to about 8 wt. %, or any range or subrange thereof, based on the total weight of the food composition on a dry weight basis.

Additionally, or alternatively, the pet food composition may include carbohydrates, e.g., in an amount up to about 65 wt. %, based on the total weight of the pet food composition on a dry matter basis. The term “carbohydrate” as used herein includes polysaccharides (e.g., starches and dextrins) and sugars (e.g., sucrose, lactose, maltose, glucose, and fructose) that are metabolized for energy when hydrolyzed. One skilled in the art could manipulate the texture of the final product by properly balancing carbohydrate sources. For example, short chain polysaccharides lend to be sticky and gluey, and longer chain polysaccharides are less sticky and gluey than the shorter chain; the desired texture of this hybrid food is achieved by longer chain polysaccharide and modified starches such as native or modified starches, cellulose and the like. The carbohydrate mixture may additionally comprise optional components such as added salt, spices, seasonings, vitamins, minerals, flavorants, colorants, and the like. The amount of the optional components is at least partially dependent on the nutritional requirements for different life stages of animals.

Carbohydrates can be supplied by any of a variety of sources known by those skilled in the art, including, but not limited to, oat fiber, cellulose, peanut hulls, beet pulp, parboiled rice, cornstarch, corn gluten meal, cereal, and sorghum. Grains supplying carbohydrates can include, but are not limited to, wheat, durum, semolina, corn, barley, and rice. In certain embodiments, the carbohydrate component comprises a mixture of one or more carbohydrate sources. Carbohydrates content of foods can be determined by any number of methods known by those of skill in the art.

Generally, carbohydrate percentage can be calculated as nitrogen free extract (“NFE”), which can be calculated as follows: NFE %=100%−(moisture %)−(protein %)−(fat %)−(ash %)−(crude fiber %) The amount of carbohydrate, e.g., calculated as NFE, present in the composition may be from an amount up to about 65 wt. %, an amount up to about 60 wt. %, an amount up to about 55 wt. %, an amount up to about 50 wt. %, an amount up to about 45 wt. %, an amount up to about 40 wt. %, an amount up to about 35 wt. %, an amount up to about 30 wt. %, an amount up to about 25 wt. %, an amount up to about 20 wt. %, an amount up to about 15 wt. %, an amount up to about 10 wt. %, an amount up to about 5 wt. %; about 1 to about 65 wt. %, about 1 to about 55 wt. %, about 1 to about 50 wt. %, about 1 to about 45 wt. %, about 1 to about 40 wt. %, about 1 to about 35 wt. %; about 1 to about 30 wt. %, about 1 to about 25 wt. %, about 1 to about 20 wt. %, about 1 to about 15 wt. %, about 1 to about 10 wt. %, about 1 to about 5 wt. %; about 5 to about 65 wt. %, about to about 55 wt. %, about 5 to about 50 wt. %, about 5 to about 45 wt. %, about 5 to about 40 wt. %, about 5 to about 35 wt. %; about 5 to about 30 wt. %, about 5 to about 25 wt. %, about 5 to about 20 wt. %, about 5 to about 15 wt. %; about 10 to about 65 wt. %, about 10 to about 55 wt. %, about 10 to about 50 wt. %, about 10 to about 45 wt. %, about 10 to about 40 wt. %, about 10 to about 35 wt. %; about 10 to about 30 wt. %, about 10 to about 25 wt. %; about 15 to about 65 wt. %, about 15 to about 55 wt. %, about 15 to about 50 wt. %, about 15 to about 45 wt. %, about 15 to about 40 wt. %, about 15 to about 35 wt. %; about 15 to about 30 wt. %; about 20 to about 65 wt. %, about 20 to about 55 wt. %, about 20 to about 50 wt. %, about 20 to about 45 wt. %, about 20 to about 40 wt. %, about 20 to about 35 wt. %; about 25 to about 65 wt. %, about 25 to about 55 wt. %, about 25 to about 50 wt. %, about 25 to about 45 wt. %, about 25 to about 40 wt. %, about 25 to about 35 wt. %; about 30 to about 65 wt. %, about 30 to about 55 wt. %, about 30 to about 50 wt. %, about 30 to about 45 wt. %; about 35 to about 65 wt. %, about 35 to about 55 wt. %, about 35 to about 50 wt. %; about 40 to about 65 wt. %, about 40 to about 55 wt. %, about 45 to about 65 wt. %, about 45 to about 55 wt. %; or about 50 to about 65 wt. %, including ranges and subranges thereof, based on the total weight of the pet composition on a dry matter basis. In some cases, the pet food composition is formulated to have about 27 to 49 wt. % of carbohydrate, based on the total weight of the pet food composition on a dry matter basis.

In certain embodiments, the pet food composition comprises moisture. The moisture may be present at various amounts or concentrations. In one embodiment, moisture may be present in an amount of from about 3 to about 20 wt. %, based on the total weight of the pet food composition. For example, moisture may be present in an amount of about 3 wt. %, about 5 wt. %, about 5.5 wt. %, about 6 wt. %, about 6.5 wt. %, about 7 wt. %, about 7.5 wt. %, about 8 wt. %, about 8.5 wt. %, about 9 wt. %, about 9.5 wt. %, about 10 wt. %, about 10.5 wt. %, about 11 wt. %, about 11.5 wt. %, about 12 wt. %, about 12.5 wt. %, about 13 wt. %, about 13.5 wt. %, about 14 wt. %, about 14.5 wt. %, or about 15 wt. %, based on the total weight of the pet food composition. In another example, moisture may be present in an amount of from about 6% to about 12%, about 9% to about 13%, about 9% to about 11%, or about 9% to about 13%, based on the total weight of the pet food composition. In certain embodiments, moisture is present in an amount of about 5% to about 12%, about 6% to about 11%, or about 7% to about 10.0%, based on the total weight of the pet food composition. In further embodiments, moisture is present in an amount of about 65% to about 85%, about 60% to about 80%, or about 60% to about 75%, based on the total weight of the pet food composition.

The pet food compositions may include one or more ingredients and/or sources of glucose mimetic, carotenoids, and/or arginine and derivatives thereof. Sources of glucose mimetics may comprise glucose anti-metabolites including 2-deoxy-D-glucose, 5-thio-D-glucose, 3-O-methylglucose, anhydrosugars including 1,5-anhydro-D-glucitol, 2,5-anhydro-D-glucitol, and 2,5-anhydro-D-mannitol, mannoheptulose, and/or avocado extract comprising mannoheptulose. Sources of carotenoids may include lutein, astaxanthin, zeaxanthin, bixin, lycopene, and/or beta-carotene. Sources of antioxidant ingredients may comprise tocopherols (vitamin E), vitamin C, vitamin A, plant-derived materials, carotenoids (described above), selenium, and/or CoQ10 (Co-enzyme Q10). In a preferred embodiment, the pet food composition contains high levels of arginine and derivatives thereof. The amount of arginine present in the composition may be about to about 10.0 wt. %, about 0.01 to about 5.0 wt. %, about 0.01 to about 2.0 wt. %, about 0.1 to about 10.0 wt. %, about 0.1 to about 5.0 wt. %, about 0.1 to about 2.0 wt. %; about 0.5 to about 5.0 wt. %, about 0.5 to about 2.0 wt. %, about 1.5 to about 5.0 wt. %, about 1.5 to about 2.0 wt. %, about wt. %, about 1.0 wt. %, about 1.4 weight %, about 1.44 wt. %, about 1.8 wt. %, or about 2.0 wt. %, based on the total weight of the composition on a dry matter basis. The arginine present in the composition may be L-arginine, D-arginine, or a mixture thereof.

The pet food compositions disclosed herein may be wet or dry compositions, and the ingredients can be either incorporated into the food composition and/or on the surface of any composition component, such as, for example, by spraying, agglomerating, dusting, or precipitating on the surface. Additionally, the pet food compositions may be formulated and produced to be in various forms and/or consistencies. For instance, the pet food compositions may, for example, be a dry, moist or semi-moist animal food composition. “Semi-moist” refers to a food composition containing from about 25 to about 35% moisture. “Moist” food refers to a food composition that has a moisture content of about 60 to 90% or greater. “Dry” food refers to a food composition with about 3 to about 12% moisture content and is often manufactured in the form of small bits or kibbles.

The food products may also include components of more than one consistency, for example, soft, chewy meat-like particles or pieces as well as kibble having an outer coating and an inner “core” component. In some embodiments, the pet food compositions may be in the form of a kibble or food kibble. As used herein, the term “kibble” or “food kibble” refers to a particulate pellet, e.g., like a component of feline or canine feeds. In some embodiments, a food kibble has a moisture, or water, content of less than 15% by weight. Food kibbles may range in texture from hard to soft. Food kibbles may range in internal structure from expanded to dense. Food kibbles may be formed by an extrusion process or a baking process. In non-limiting examples, a food kibble may have a uniform internal structure or a varied internal structure. For example, a food kibble may include a core and a coating to form a coated kibble. It should be understood that when the term “kibble” or “food kibble” is used, it can refer to an uncoated kibble or a coated kibble.

The composition of the present disclosure can additionally comprise other additives in amounts and combinations familiar to one of skill in the art. Such additives should be present in amounts that do not impair the purpose and effect provided by the invention. Examples of additives include substances with a stabilizing effect, organoleptic substances, processing aids, and substances that provide nutritional benefits.

Stabilizing substances may include, by way of example, substances that tend to increase the shelf life of the pet food composition. Other examples of other such additives potentially suitable for inclusion in the compositions of the invention include, for example, preservatives, antioxidants, synergists and sequestrants, packaging gases, stabilizers, emulsifiers, thickeners, gelling agents, and humectants. Examples of emulsifiers and/or thickening agents include gelatin, cellulose ethers, starch, starch esters, starch ethers, and modified starches. Additives for coloring, palatability, and nutritional purposes can include colorants, salts (including, but not limited to, sodium chloride, potassium citrate, potassium chloride, and other edible salts), vitamins (e.g., Vitamin B, Vitamin C, and/or Vitamin E), minerals, and flavoring. For example, in some embodiments, the pet food composition includes one or more Vitamins, such as one or more vitamin B selected from thiamin, riboflavin, niacin, pantothenic acid, pyridoxine, biotin, folic acid, cobalamin and a combination of two or more thereof. In some instances, the pet food composition has at least one of Thiamin in an amount of from about 8 to about 20 mg/100 g, Pyridoxine in an amount of from about 1 to about 4 mg/100 g, Cobalamin in an amount of from about 10 to about 40 ug/100 g, and Folic Acid in an amount of from about 100 to about 400 ug/100 g. The pet food composition may be formulated to have a weight ratio of folic acid to cobalamin of from about 1:1 to about 50:1, preferably about 1:1 to about 40:1, preferably about 3:1 to about 40:1, or preferably about 5:1 to about 40:1. Additionally or alternatively, the pet food composition may have a weight ratio of thiamin to pyridoxine of from about 1:1 to about 30:1, preferably about 2:1 to about 30:1, or preferably about 2:1 to about 20:1. Other additives can include glucosamine, chondroitin sulfate, vegetable extracts, herbal extracts, etc.

The concentration of such additives in the pet food composition typically can be up to about wt. %, based on the total weight of the pet food composition on a dry matter basis. For example, the additives may be present in an amount from about 0.01 to about 5 wt. %, about 0.01 to about 4 wt. %, about 0.01 to about 4 wt. %, about 0.01 to about 3 wt. %, about 0.01 to about 2 wt. %, about to about 1 wt. %; about 0.1 to about 5 wt. %, about 0.1 to about 4 wt. %, about 0.1 to about 4 wt. %, about 0.1 to about 3 wt. %, about 0.1 to about 2 wt. %, about 0.1 to about 1 wt. %; about 1 to about 5 wt. %, about 1 to about 4 wt. %, about 1 to about 4 wt. %, about 1 to about 3 wt. %, about 1 to about 2 wt. %; about 2 to about 5 wt. %, about 2 to about 4 wt. %, about 2 to about 4 wt. %, about 2 to about 3 wt. %; about 3 to about 5 wt. %, about 3 to about 4 wt. %; or about 4 to about 5 wt. %, based on the total weight of the pet food composition on a dry matter basis. In some embodiments, the concentration of such additives (particularly where such additives are primarily nutritional balancing agents, such as vitamins and minerals) is from about 0 to about 2.0% by weight, based on the total weight of the pet food composition on a dry matter basis. In some embodiments, the concentration of such additives (again, particularly where such additives are primarily nutritional balancing agents) is from about 0 to about 1.0% by weight, based on the total weight of the pet food composition on a dry matter basis. Although the list of foregoing additives may be potentially suitable in some embodiments, one or more of the foregoing additives may be excluded from other embodiments of the pet food composition.

In specific embodiments, the pet food compositions and food products are formulated to address specific nutritional differences between species and breeds of animals, as well as one of more of the attributes of the animal. For example, cat foods, for example, are typically formulated based upon the life stage, age, size, weight, body composition, and breed.

Sources of proteins, carbohydrates, fats, vitamins, minerals, balancing agents, and the like, suitable for inclusion in the pet food compositions, and particularly in the food products to be administered in methods provided herein, may be selected from among those conventional materials known to those of ordinary skill in the art.

The pet food compositions may include, or in some instance exclude, one or more ingredients selected from beef broth, brewers dried yeast, egg, egg product, flax meal, DL methionine, amino acids, leucine, lysine, arginine, cysteine, cystine, aspartic acid, polyphosphates, sodium pyrophosphate, sodium tripolyphosphate; zinc chloride, copper gluconate, stannous chloride, stannous fluoride, sodium fluoride, triclosan, glucosamine hydrochloride, chondroitin sulfate, green lipped mussel, blue lipped mussel, methyl sulfonyl methane (MSM), boron, boric acid, phytoestrogens, phytoandrogens, genistein, diadzein, Lcarnitine, chromium picolinate, chromium tripicolinate, chromium nicotinate, acid/base modifiers, potassium citrate, potassium chloride, calcium carbonate, calcium chloride, sodium bisulfate; eucalyptus, lavender, peppermint, plasticizers, colorants, flavorants, sweeteners, buffering agents, slip aids, carriers, pH adjusting agents, natural ingredients, stabilizers, biological additives such as enzymes (including proteases and lipases), chemical additives, coolants, chelants, denaturants, drug astringents, emulsifiers, external analgesics, fragrance compounds, humectants, opacifying agents (such as zinc oxide and titanium dioxide), antifoaming agents (such as silicone), preservatives (such as butylated hydroxytoluene (BHT) and butylated hydroxyanisole (BHA), propyl gallate, benzalkonium chloride, EDTA, benzyl alcohol, potassium sorbate, parabens and mixtures thereof), reducing agents, solvents, hydrotropes, solubilizing agents, suspending agents (non-surfactant), solvents, viscosity increasing agents (aqueous and non-aqueous), sequestrants, keratolytics, egg shell membrane, ancient grain, and a combination of two or more thereof.

The pet food composition may be produced by various methods to achieve the desired pet food composition or desired form for the pet food composition. For example, dry food may be baked or extruded, then cut into individual shaped portions, such as kibbles. In some embodiments, the pet food composition may be prepared in a canned or wet form using conventional food preparation processes known to those of ordinary skill in the art. Typically, ground animal proteinaceous tissues are mixed with the other ingredients, such as cereal grains, suitable carbohydrate sources, fats, oils, and balancing ingredients, including special purpose additives such as vitamin and mineral mixtures, inorganic salts, cellulose, beet pulp and the like, and water in an amount sufficient for processing. The ingredients are mixed in a vessel suitable for heating while blending the components. Heating the mixture is carried out using any suitable manner, for example, direct steam injection or using a vessel fitted with a heat exchanger. Following addition of all of the ingredients of the formulation, the mixture may be heated to a temperature of from 50° F. to 212° F. Although temperatures outside this range can be used, they may be commercially-impractical without the use of other processing aids. When heated to the appropriate temperature, the material will typically be in the form of thick liquid, which is dispensed into cans. A lid is applied and the container is hermetically sealed. The sealed can is then placed in convention equipment designed for sterilization of the contents. Sterilization is usually accomplished by heating to temperatures of greater than 230° C. for an appropriate time depending on the temperature used, the nature of the composition, and related factors. The pet food compositions and food products of the present disclosure can also be added to or combined with food compositions before, during, or after their preparation.

In some embodiments, the food products may be prepared in a dry form using convention processes known to those of ordinary skill in the art. Typically, dry ingredients, including dried animal protein, plant protein, grains and the like are ground and mixed together. Liquid or moist ingredients, including fats, oils water, animal protein, water, and the like are added combined with the dry materials. The specific formulation, order of addition, combination, and methods and equipment used to combine the various ingredients can be selected from those known in the art. For example, in certain embodiments, the resulting mixture is process into kibbles or similar dry pieces, which are formed using an extrusion process in which the mixture of dry and wet ingredients is subjected to mechanical work at high pressure and temperature, forced through small openings or apertures, and cut off into the kibbles, e.g., with a rotating knife. The resulting kibble can be dried and optionally coated with one or more topical coatings comprising, e.g., flavors, fats, oils, powdered ingredients, and the like. Kibbles may also be prepared from dough by baking, rather than extrusion, in which the dough is placed into a mold before dry-heat processing.

In preparing a composition, any ingredient generally may be incorporated into the composition during the processing of the formulation, e.g., during and/or after mixing of the other components of the composition. Distribution of these components into the composition can be accomplished by conventional means. In certain embodiments, ground animal and/or poultry proteinaceous tissues are mixed with other ingredients, including nutritional balancing agents, inorganic salts, and may further include cellulose, beet pulp, bulking agents and the like, along with sufficient water for processing.

In accordance with another aspect, provided is a method for reducing metabolites associated with protein putrefaction in a feline, the method including reducing one or more proteolytic metabolites and increasing one or more short chain fatty acid in a feline by administering a pet food composition described herein to the feline. In some embodiments, the one or more proteolytic metabolites comprises pyrraline, N6-carboxymethyllysine, N6-carboxyethyllysine, methionine sulfoxide, pseudouridine, or a combination of two or more thereof. The method may include providing and/or feeding the feline the pet food compositions for 1 or more days, preferably 5 or more days, preferably 7 or more days, preferably 10 or more days, preferably 14 or more days, preferably 30 or more days, or preferably 42 or more days. The method may include feeding the feline one time a day, two times a day, three times a day, or in some embodiments four or more times a day.

### EXAMPLES

The following non-limiting examples are provided primarily for the purposes of elucidating the benefits and properties achieved by aspects of the invention.

### Example 1

A non-limiting, exemplary pet food composition (Example Composition A) and a comparative pet food composition (Comparative Composition 1) were prepared in accordance with aspects of the invention. The formula of Example Composition A and Comparative Composition 1 are shown in Table 1, provided below. Table 2 provides a summary of the nutritional values of Example Composition A and Comparative Composition 1.

TABLE 1

| | Ex. A | Comp. 1 |
|---|---|---|
| Ingredients | (wt. %) | (wt. %) |
| Corn gluten and Wheat gluten | 22-27 | 22-27 |
| Chicken Dried (10 wt. % of Ash) | 9-12 | 9-12 |
| Lactic acid (84 wt. % of lactic acid) | 1-1.5 | 1-1.5 |
| Soybean oil, crude, degummed | 0.3-0.6 | 0.3-0.6 |
| Beet, pulp, pelleted | | 2.5 |
| Choline chloride (liquid 70 wt. % of | 0.25-0.5 | 0.25-0.5 |
| choline chloride) | | |
| Fish oil | 0.9-1.2 | 0.9-1.2 |
| Carnitine, 1 (10 wt. % of Carnitine) | 0.3-0.6 | 0.3-0.6 |
| Taurine | 0.1-0.3 | 0.1-0.3 |
| Vitamin Premix, Mineral Premix, and | 0.4-0.6 | 0.4-0.6 |
| Vitamin E oil (29 wt. % of Vit. E) | | |
| Wheat (whole) and Rice (brewers) | 35-43 | 35-43 |
| Pork Fat, Choice White Grease | 10-14 | 10-14 |
| Egg Dried pelleted | 4.5-6 | 4.5-6 |
| Chicken Liver Digest | 2-2.7 | 2-2.7 |
| Flax seed whole brown | 2-2.6 | 2-2.6 |
| Potassium Chloride and | 0.6-0.9 | 0.6-0.9 |
| Sodium Chloride | | |
| Calcium Carbonate and Calcium Sulfate | 1-1.5 | 1-1.5 |
| Oat Fiber, Fruit, Vegetable blend | | 0.04 |
| Total Components | 100 | 100 |

TABLE 2

| | Ex. A | Comp. 1 |
|---|---|---|
| Protein (%) | 30.86 | 32.71 |
| Crude fiber (%) | 0.9 | 1.4 |
| Protein:Fiber | 34.3 | 23.4 |
| Ratio | | |

### Example 2

Example Composition A was evaluated in comparison to Comparative Composition 1 by feeding both pet food compositions to a group of felines. Specifically, a group of forty cats between the age of 8.3 and 13.5 years were fed Example Composition A for a first period of four weeks. After the first four-week period, the felines were then fed Comparative Composition 1 for a second period of four weeks.

Fecal samples were collected from each of the felines at the end of each four-week feeding period to compare levels of putrefactive metabolites. The mean differences in the concentrations of various proteolytic and oxidative metabolites after the felines were fed the pet food compositions was calculated by subtracting the concentration of the proteolytic and oxidative metabolites assessed after the 4 weeks period of feeding Comparative Composition 1 from the concentration of the proteolytic and oxidative metabolites assessed after the 4 weeks period of feeding Example Composition A (mean difference=Example Composition A−Comparative Composition 1). Table 3, shown below, provides the determined concentrations of the proteolytic and oxidative metabolites and the calculated mean difference in such concentrations based on the felines receiving Example Composition A and Comparative Composition 1.

TABLE 3

| | Mean Δ | | |
|---|---|---|---|
| Metabolite | (Ex. A − Comp. 1) | P-Value | SE |
| kynurenine | −0.27 | 0.00025 | 0.067 |
| glycylleucine | −0.37 | 0.00036 | 0.092 |
| valylleucine | −0.63 | 0.00046 | 0.16 |
| isoleucylglycine | −0.6 | 0.00058 | 0.16 |
| glycylvaline | −0.33 | 0.001 | 0.093 |
| valylglycine | −0.47 | 0.001 | 0.13 |
| tryptophylglycine | −0.5 | 0.004 | 0.16 |
| glutamine | −0.24 | 0.0044 | 0.078 |
| N6-formyllysine | −0.3 | 0.005 | 0.1 |
| leucylglycine | −0.5 | 0.0053 | 0.17 |
| threonylphenylalanine | −0.5 | 0.0061 | 0.17 |
| methionine | −0.21 | 0.0063 | 0.071 |
| serine | −0.22 | 0.0065 | 0.076 |
| N-methylproline | −0.63 | 0.0067 | 0.22 |
| glutaminylleucine | −0.35 | 0.0069 | 0.12 |
| glycylisoleucine | −0.29 | 0.0098 | 0.11 |
| N-methylalanine | −0.31 | 0.013 | 0.12 |
| gamma-glutamylleucine | −0.35 | 0.014 | 0.14 |
| phenylalanylglycine | −0.32 | 0.016 | 0.13 |
| leucylalanine | −0.33 | 0.02 | 0.14 |
| Leucylglutamine | −0.26 | 0.023 | 0.11 |
| phenylalanine | −0.13 | 0.035 | 0.059 |
| leucine | −0.13 | 0.038 | 0.062 |
| alanylleucine | −0.23 | 0.039 | 0.11 |
| N,N,N-trimethyl-5- | −0.23 | 0.00034 | 0.058 |
| aminovalerate | | | |
| kynurenate | −0.17 | 0.017 | 0.068 |
| isovalerate (C5) | −0.43 | 0.00024 | 0.1 |
| 3-methyl-2-oxovalerate | −0.3 | 0.016 | 0.12 |

Table 4 provides the determined concentration of oxidative metabolites and the calculated mean difference in such concentrations based on the felines receiving Example Composition A and Comparative Composition 1.

TABLE 4

| | | Mean Δ | | |
|---|---|---|---|---|
| Metabolite | P-value | (Ex. A − Comp. 1) | P-value | SE |
| pyrraline | 2.3E−24 | −1.6 | 2.3E−24 | 0.058 |
| N6-carboxy- | 0.00044 | −0.36 | 0.00044 | 0.092 |
| methyllysine | | | | |
| N6-carboxy- | 0.0025 | −0.37 | 0.0025 | 0.11 |
| ethyllysine | | | | |
| methionine | 0.0048 | −0.24 | 0.0048 | 0.079 |
| sulfoxide | | | | |
| pseudouridine | 0.021 | −0.21 | 0.021 | 0.088 |

Example Composition A advantageously leads to reduction in multiple proteolytic and oxidative metabolites in comparison to Comparative Composition 1. Additionally, Example Composition A increased beneficial short chain fatty acids, such as butyrate and butyrylglycine, while simultaneously reducing the undesirable proteolytic and oxidative metabolites as compared to Comparative Composition 1.

1. A pet food composition comprising:
a fat;
from about 15 to about 55 wt. % of a protein;
from about 0.1 to about 5 wt. % of a crude fiber; and
optionally, a carbohydrate,
wherein the pet food composition has a weight ratio of the protein to the crude fiber of from about 25:1 to about 50:1, and all weight percentages are based on the total weight of the pet food composition.

2. The pet food composition according to claim 1, wherein the weight ratio of the protein to the crude fiber is from about 28:1 to about 45:1.

3. The pet food composition according to claim 1, wherein the weight ratio of the protein to the crude fiber is from about 30:1 to about 38:1.

4. The pet food composition according to any foregoing claim 1, wherein the weight ratio of the protein to the crude fiber is from about 32:1 to about 36:1.

5. The pet food composition according to any foregoing claim 1, wherein the crude fiber comprises a soluble fiber and an insoluble fiber.

6. The pet food composition according to any foregoing claim 1, wherein the fat is present in an amount of from about 8 to about 50 wt. %.

7. The pet food composition according to any foregoing claim 1, wherein the fat is present in an amount of from about 15 to about 25 wt. %.

8. The pet food composition according to claim 1, wherein the pet food composition further comprises one or more short chain fatty acid.

9. The pet food composition according to claim 1, wherein the protein comprises one or more amino acids selected from glycine, proline, tryptophan, taurine, histidine, carnitine, carnosine, alanine, cysteine, arginine, methionine, lysine, asparagine, aspartate and/or aspartic acid, phenylalanine, valine, threonine, isoleucine, histidine, leucine, glutamine, taurine, tyrosine, homocysteine, ornithine, citruline, glutamate and/or glutamic acid, serine, and a combination of two or more thereof.

10. The pet food composition according to claim 9, wherein the protein comprises glycine, proline, or a combination of two or more thereof.

11. The pet food composition according to claim 1, wherein the carbohydrate is present in an amount up to about 65 wt. %.

12. The pet food composition according to claim 1, wherein the carbohydrate is present in an amount from about 27 to about 49 wt. %.

13. A pet food composition comprising:
from about 8 to about 50 wt. % of a fat;
from about 15 to about 55 wt. % of a protein;
from about 0.1 to about 5 wt. % of crude fiber; and
an amount up to about 65 wt. % of a carbohydrate,
wherein the pet food composition has a weight ratio of the protein to the crude fiber of from about to about 50:1, and all weight percentages are based on the total weight of the pet food composition.

14. The pet food composition according to claim 13, wherein the weight ratio of the protein to the crude fiber is from about 30:1 to about 38:1.

15. The pet food composition according to claim 13, wherein the weight ratio of the protein to the crude fiber is from about 32:1 to about 36:1.

16. A method for reducing metabolites associated with protein putrefaction in a feline, the method comprising: reducing one or more proteolytic metabolites and increasing one or more short chain fatty acid in a feline by administering a pet food composition according to claim 13 to the feline.

17. The method according to claim 16, wherein the one or more short chain fatty acid comprises butyrate.

18. The method according to claim 16, wherein the one or more proteolytic metabolites comprises pyrraline, N6-carboxymethyllysine, N6-carboxyethyllysine, methionine sulfoxide, pseudouridine, or a combination of two or more thereof.

19. The method according to claim 16, wherein the feline is fed the pet food composition at least once a day, preferably at least twice a day, or preferably at least three times a day.

20. The method according to claim 16, wherein the feline is fed the pet food composition for 1 or more days.
